(19) United States
(12) Patent Application Publication
(10) Pub. No.: US 2009/0260271 A1
(43) Pub. Date: Oct. 22, 2009

(54) Visible animal scent and dispersion system

(76) Inventor: Don Bailey, Mead, WA (US)

Correspondence Address: BERGMAN & JECKLE P.L.L.C., 421 W. RIVERSIDE, SUITE 717, SPOKANE, WA, 99201, US

(21) Appl. No.: 12/082,867

(22) Filed: Apr. 16, 2008

(51) Int. Cl. A01M 31/00; A61K 9/14

(52) U.S. Cl. 43/1; 424/46

(57) ABSTRACT

An animal scent composition contains animal generated scents and a visual indicator to allow visualization of scent travel and application. The scent may be used for repelling wild and domestic animals, for attracting game animals, and for masking the scent of humans. Dispersing means provide a pressurized aerosol canister with a dispersing nozzle axially aligned with the aerosol canister to prevent clogging of the nozzle by agglomerated visual indicator particles when the scent composition is dispersed.

### II. BACKGROUND OF INVENTION

### IIA. RELATED APPLICATIONS

There are no applications related hereto heretofore filed in this or in any foreign country.

### IIB. FIELD OF INVENTION

My invention relates generally to visible aerosol scents used to influence the behavior of animals and dispersing means therefore.

### IIC. DESCRIPTION OF PRIOR ART

Animals generally have sensory and physical abilities beyond such abilities of humans. The physical abilities, such as strength, speed, and stamina are obvious, but sensory abilities such as smell, vision, hearing, and arguably instinct, though less obvious, are equally beyond similar human abilities.

In particular the olfactory senses of animals are more advanced and sensitive than those of humans, and the environment animals live in is dominated by odors to an extent humans cannot know nor experience. For example, it has been estimated that a domestic dog's sense of smell is between 1,000 to 10,000 times as sensitive as that of a human.

Animals communicate by odiferous means and are well aware of one another's presence, and past presence, even when not in audible or visual contact with one another, by the odors they produce. These odors may originate from urine, feces and glands such as the tarsal, metatarsal, interdigital and preorbital. Other odors are produced during various activities such as ovulation and copulation. These odors are a means for communicating with other animals, give the ability to locate one another to form herds for protection, socialization and feeding, to mark territory and for various other purposes including propagation of the species.

Animals may react to some scents positively and other scents negatively. Reactions may be hormonally driven, genetically predetermined, instinctual, trained, or caused by some combination of these reactions. Humans have capitalized on these reactions and have long attempted to use the effluvia of odors and scents to influence animal behavior with the goal of making sensing animals respond as desired.

Animal scents may be employed by hunters and by nature and wildlife photographers to come into close proximity to wildlife to photograph or harvest animals. Animal scents may also be used by various state and governmental conversation personnel and biologists who need to attract or capture animals for health checks, disease control, reproductive measurements, game population control and relocation programs.

Animal scents, which are generally quite odoriferous and often offensive to humans, are often used as attractants, scent masking agents and repellents. Commonly, natural scent may be obtained from the urine of an animal. When an attractant is sought, the urine may be obtained from a female animal in estrus, which contains pheromones that are attractive to males of the species. Attractants, and scent masking agents are most commonly employed by hunters, wildlife photographers and game biologists when seeking game animals such as deer, elk, bear, moose and the like. By application of the scent, the hunter, photographer or biologist intends to both attract the animal and mask or cover up human scent with an odor that is attractive and appealing to the animal so as to lure the animal into proximity to the person and overcome the animal's otherwise cautious nature and human scent aversion.

Animal repellants, which are also often formulated with animal urine, are used to keep domestic and wild animals away from crops, gardens, flower beds, orchards, lawns and the like. Use of animal urine in repellants is one of the novel aspects of the instant invention as the inventor's research has shown domestic dogs and wild deer, will vigorously avoid areas where tiger or other large cat urine has been dispersed, even though the domestic dog has never come into contact with a tiger or large cat.

The use of animal scents, however, is not without inherent difficulties. Commonly the scent is a relatively clear liquid and is invisible once dispersed. This invisibility often leads to saturation in one area while failing to disperse scent in an adjoining area, difficulty insuring the scent is dispersed where intended, difficulty of the disperser in avoiding being covered in the animal scent, determining where the scent is carried by the wind after dispersal, dispersing the scent without the need to physically handle it, and not contaminating the animal scent with human scent.

Various methods and devices for dispersing animal scent into a surrounding area are disclosed in the prior art. Most such methods and devices relate to dispersal of animal attractants in an effort to bring game animals into close proximity to hunters. Such methods of dispersing game attractants generally fall into three categories: drip dispersion, vaporization, and manual dispersion. These methods may or may not be used in combination with other methods of attracting game animals such as noise makers, decoys or the like.

I have found that an aerosol canned animal scent with finely particulated diatomaceous earth, cornstarch or both can be seen visually when sprayed down wind toward animals that are to be attracted as this particulate matter may be small enough to float with the wind and remain airborne for substantial periods of time toward animals down wind. Avoiding contact with the scent is nearly impossible however when low pressure squeeze bottles, or open reservoirs are used because of the need to physically handle the scent or containers to operate reservoirs, squeeze bulbs, or drip dispersers. Physically handling the animal scent often leads to the operator contamination with scent which generally is offensively odoriferous and long lasting.

Determination of wind direction is important in dispersing animal scent and various methods and devices for determining wind direction are disclosed in the prior art. These devices generally fall into two categories: mechanical and free floating particulate material. Mechanical means are characterized by filaments, vanes or other mechanical indicators that move in the wind to indicate wind direction. “Free floating” indicators are characterized by small particulates such as talc, handfuls of grass, dust, leaves or the like, which are dispersed or thrown into the air. Movement of the particles in the wind is visualized to determine the wind direction but their airborne state may not be very long lasting.

To be effective, such particulate material must not be alarming to the animal by smell, vision or otherwise. Determining wind direction by use of particulate material is often ineffective because an animal frequently may see or otherwise sense the action required to disperse the particulate material. Dispersing talc, diatomaceous earth or similar visible particulate material with pressurized aerosol canisters is efficient and reliable because of the relatively small particulates that may be obtained which do not clog the aperture of aerosol dispersal containers and the lack of unnatural odor which may alert animals, especially if not masked with some other scent. Clogging of the aerosol dispersement apertures may make particulate dispersing devices inoperative, or at least not consistently operative. Inconsistent operation is exacerbated by environmental conditions such as high humidity and low temperatures which are common in outdoor environs. Particulate material may be dispersed with a low pressure squeeze bulb device having a larger g aperture but controlling the amount of material dispersed becomes problematic. Further, the ability to make multiple dispersions to repeatedly check wind direction is desirable as wind direction and localized air currents and eddies change with temperature and terrain changes.

It can be appreciated to one skilled in the art that there exists a continuing need for a new and improved method and apparatus for dispersing animal behavior influencing scent with a visual flow indicator and a reliable dispersing means which can be used by hunters and other users. The present invention seeks to fulfill this need.

The present system departs from the conventional concepts and designs of the prior art by providing readily dispersible animal scent compositions which may be used to repel wild and domestic animals from areas where they cause damage or are a nuisance to attract game animals and to mask the scent of hunters, photographers or the like. The present scent compositions provide a novel particulate indicator to allow visual detection of scent dispersement as well as determination of wind direction and localized wind currents and eddies and further provides an aerosol apparatus for efficient and consistently reliable dispersion of the visually detectible animal scent.

The novel use of corn starch, diatomaceous earth or both in the present system provides a visual indicator for the scent composition but tends to prevent use of common pressurized aerosol containers having nozzles that disperse perpendicular to the axis of the container. Both corn starch and diatomaceous earth have been found to act as an absorbent of animal scent. The animal scent material is carried by these particles for a period of time before the animal scent volatizes and becomes airborne after dispersal from the aerosol canister. While intermixed, the particles of visual material and the liquid tend to agglomerate and are large enough to clog the opening aperture of standard perpendicularly dispersing aerosol nozzles. Therefore a critical component of the present invention is use of an aerosol valve assembly that disperses the instant composition parallel to the axis of the aerosol canister.

The use of corn starch, diatomaceous earth or both further provides an additional novel feature in that when the scent composition is dispersed, the odoriferous nature of the scent does not become noticeable for a delayed period of usually about 30 second subsequent to dispersal depending on local environmental conditions. This time lag allows an operator to visualize the dispersal and wind direction, including local currents and eddies, before the scent regains its odiferous nature.

### III. SUMMARY OF INVENTION

My invention generally provides a visible animal scent composition and dispersing means therefore. The scent composition provides a mixture of “DYMEL”® 134a/P, a product of DuPont Corporation, animal urine, corn starch, diatomaceous earth or both and a carrier such as isopropyl alcohol. Urine from a female animal in estrus may be used as an attractant and as a scent masking agent for males of the species and urine of large felines such as tigers, lions, or leopards may be used as a repellant for dogs, deer, elk or the like.

Dispersion of the composition is made with a pressurized aerosol canister with an opening aperture oriented parallel to the axis of the canister to be operated by a finger pressure valve.

In providing such a system it is a principal object to provide an animal scent composition of “DYMEL”® 134a/P a product of DuPont Corporation, animal urine, corn starch, diatomaceous earth or both and isopropyl alcohol to attract or repel animals and mask the scent of a human.

A further object is to provide a pressurized aerosol canister with a valved aperture parallel to the axis of the canister to permit passage of the animal scent, liquid and propellant therethrough without clogging the aperture.

A further object of the instant invention is to use corn starch, diatomaceous earth or both as a visible adsorbent agent in the scent composition which carries and represses the scent composition at and shortly after dispersal.

A still further object is to use corn starch, diatomaceous earth or both a visual indicator which remain opaque in various weather conditions to allow determination of wind direction, place of application of scent and motion of the scent composition in localized air currents and eddies.

A still further object is to provide a simple, economical and reliable aerosol dispersing means for animal scent compositions that allows the operator to avoid physical contact with the composition.

Other and further objects of my invention will appear from the following specification and accompanying drawings which form a part hereof. In carrying out the objects of my invention, however, it is to be remembered that its features are susceptible of change in design and structural arrangement with only one preferred and practical embodiment of the best known mode being illustrated in the accompanying drawings and specified as is required.

### IV. BRIEF DESCRIPTIONS OF DRAWINGS

In the accompanying drawings which form a part hereof and wherein like numbers of reference refer to similar parts throughout:

FIG. 1 is an orthographic view of my animal scent composition dispersing device in use to determine wind direction, local air currents and place of dispersement.

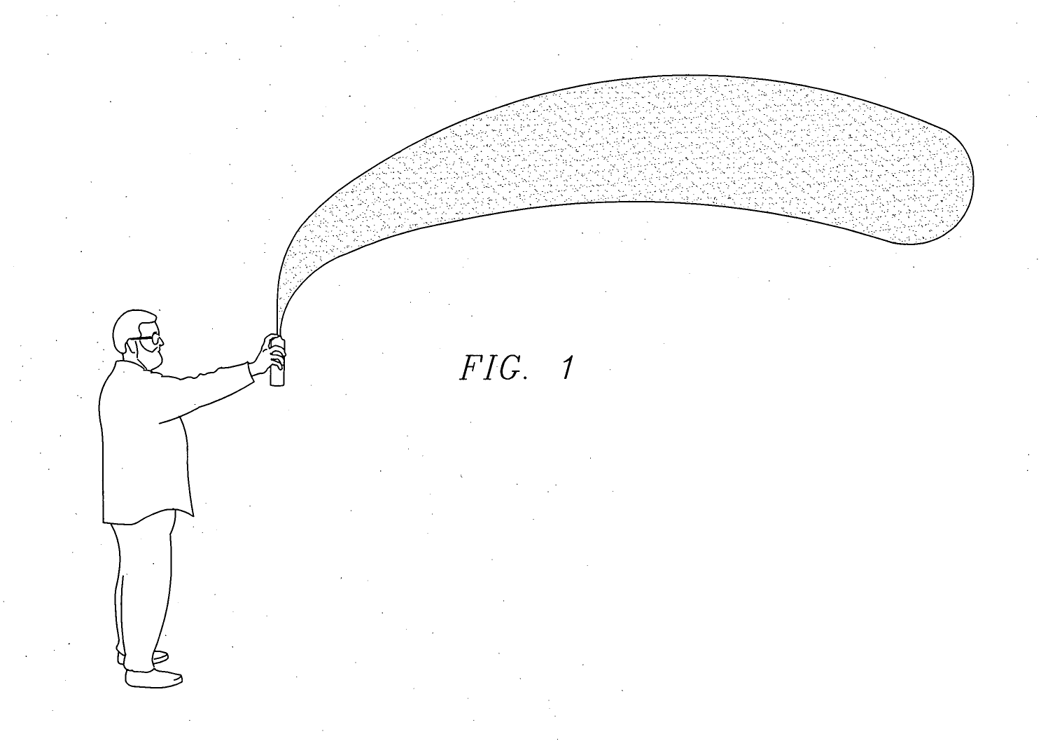

FIG. 2 is an enlarged partially cut-away isometric view of a pressurized aerosol canister with axially aligned aperture for dispersing my animal scent composition.

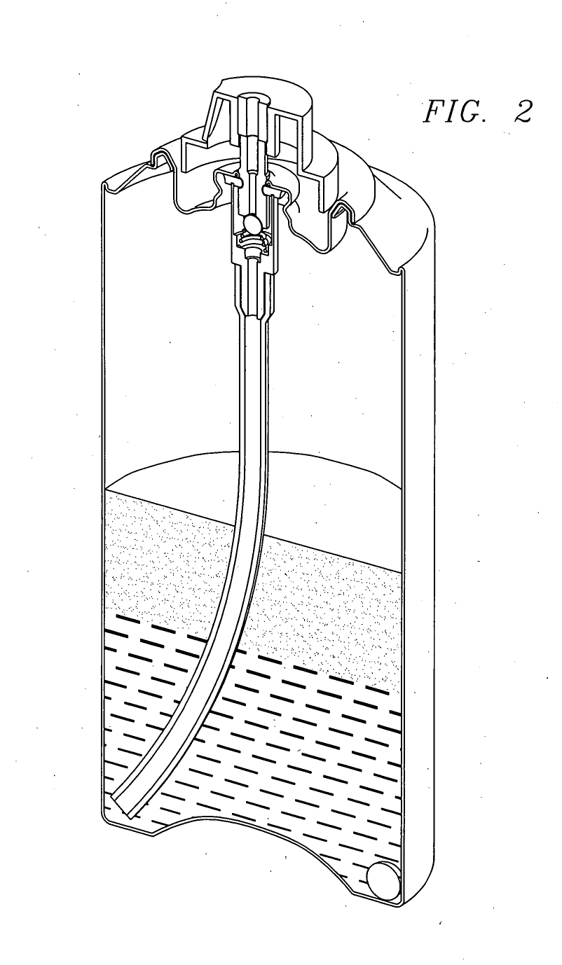

### V. DESCRIPTION OF PREFERRED EMBODIMENT

My invention generally provides a system having a scent composition that simulates an animal odor and dispersing means therefore. The scent composition contains an odiferous element, a visual indicator, a carrier and a preservative.

The scent composition may be other natural animal scents or extracts of either, especially such as a female animal in estrus or a large cat such as a tiger, lion or leopard, and comprises approximately 9.6% by weight of the composition.

The preservative may be isopropyl alcohol which comprises approximately 19.2% by weight of the composition.

The visual indicator is finely particulated corn starch, diatomaceous earth or both which collectively comprise approximately 25% by weight of the composition.

The carrier may be DYMEL® 134a/P a product of the DuPont Corporation of Wilmington, Del. which comprises approximately 46.2% by weight of the composition.

The animal scent dispersing means provides pressurized aerosol canister 1. The pressurized aerosol canister 1 is of the known type that carries at one of its ends finger operated dispersing nozzle 2 with aperture 3 oriented parallel to the axis of the pressurized aerosol canister 1, so that when activated by finger pressure fluidic content 4 of the canister 1 are dispersed in a direction parallel to the axis of the pressurized canister 1.

A supply of the before described animal scent composition is contained within the pressurized aerosol canister 1. A dispersing nozzle feed tube 5 communicates between the dispersing nozzle 2 and the fluidic content 4 of canister 1. A free moving agitator 6 is carried within the pressurized canister 1. The opening of aperture 3 in the dispersing nozzle 2 is of sufficient size to permit free passage of agglomerated corn starch particles, diatomaceous earth particles or both, pressurized gas, and liquid therethrough without clogging the aperture 3 while producing an airborne and supported mist-like spray.

Having described the composition of my animal scent and structure of its dispersing means, the operation of my system may be understood.

The method of the present invention, when being used for dispersement of a repellant, is carried out by dispersing a quantity of the fluidic content 4 of canister 1 onto objects such as rocks, trees, bushes or the ground. While the fluidic content 4 is being dispersed through * dispersing nozzle 2 of the aerosol canister 1, the operator will be able to visualize the placement of the fluidic content 4 at the selected location, while at the same time observing the air movement around the dispersal area. Verification of placement is especially critical where the fluidic content 4 is being used as a repellant. It is necessary to well cover certain areas such as animal paths, while at the same time avoiding direct application of the scent to other areas, such as plants and crops. Visualization of the fluidic content 4 during dispersion permits the operator to verify that the fluidic content 4 has been appropriately applied where intended.

The method of the present invention when being used for dispersement of either an attractant or repellant fluidic content 4 as a masking scent is firstly to disperse small amounts of the fluidic content 4 to determine wind direction and the direction of local air currents and eddies. To accomplish this dispersing nozzle 2 is depressed by an operator's finger to release a small amount of the pressurized fluidic content 4, which is visualized by an operator to determine wind activity and direction. If the wind characteristics are not as the operator desires, the operator may move to find a location where the wind direction and local air currents and eddies are as near the desired characteristics as possible. Repeated releases of small amounts of fluidic content 4 with visual indicator may be required. Repeated releases will not impair the operator's probability of success as any human scent of the operator will be overwhelmed and masked by the animal scent. Once a suitable location is found, the operator may repeatedly disperse desired amounts of the fluidic content 4 to create a scent trail which may lure the desired animal into close proximity for photographs or harvesting or repel it from the object environment. Alternatively, the operator may disperse large quantities of the fluidic content 4 in gross to try to attract animals in general to a desired area.

The foregoing description of my invention is necessarily of a detailed nature so that a specific embodiment of its best mode might be set forth as required, but it is to be understood that various modifications of details, rearrangement and multiplication of parts might be resorted to without departing from its spirit, essence, or scope.

Having thusly described my invention, what I desire to protect by Letters Patent, and

What I claim is:

1. A visible, aerosol dispersible scent composition that simulates a natural body scent of a first animal to induce behavior of a second animal containing an odiferous portion derived from glandular and alimentary excrement of an animal, an air supportable particulate visual indicator, a carrier and a preservative, the odiferous portion comprising from 4.6% to 14.6% by weight of the scent composition.

2. The composition of claim 1 wherein the preservative is isopropyl alcohol comprising 14.2% to 14.6% by weight of the scent composition.

3. The composition of claim 1 wherein the visual indicator comprises between 20% to 30% by weight of the composition and consists of:
0 to 30% weight percent of corn starch and
0 to 30% weight percent of diatomaceous earth.

4. The composition of claim 1 wherein the carrier is DYMEL® 134a/P comprising 41.2% to 51.2% by weight of the composition.

5. A visible, aerosol dispersible scent composition that simulates a natural body scent of a first animal to induce behavior of a second animal containing an odiferous portion derived from glandular and alimentary excrement of an animal, an air supportable particulate visual indicator, a carrier and a preservative comprising in combination:
41.2% to 51.2% by weight “DYMEL”® 134a/P;
4.6% to 14.6% by weight animal body scent consisting of glandular and alimentary excrement;
20% to 30% by weight visual indicator consisting of 0 to 30% of each of air supportable particulate corn starch and diatomaceous earth; and
14.2% to 24.2% by weight isopropyl alcohol.

6. The animal scent composition of claim 5 wherein
the animal body scent is urine from a female animal in estrus.

7. The animal scent composition of claim 5 wherein
the animal body scent is urine from large non-domesticated felines.

8. A means for the dispersing animal scent of claim 5 comprising:
a pressurized aerosol canister
having a finger operable dispersing nozzle with an aperture opening oriented parallel to the axis of the pressurized aerosol canister of size sufficient to permit free passage of the scent composition and any contained agglomerated particulate visual indicator therethrough.
